(19) United States
(12) Patent Application Publication
(10) Pub. No.: US 2006/0121587 A1
(43) Pub. Date: Jun. 8, 2006

(54) Process for actinol production from ketoisophorone

(76) Inventors: Tatsuo Hoshino, Kanagawa (JP); Yutaka Setoguchi, Kanagawa (JP); Sakayu Shimizu, Kyoto (JP); Kazuyuki Tabata, Kanagawa (JP)

Correspondence Address: Stephen M Haracz;Bryan Cave, 1290 Avenue of the Americas, New York, NY, 10104, US

(21) Appl. No.: 10/528,843

(22) PCT Filed: Sep. 16, 2003
(86) PCT No.: PCT/EP03/10295

§ 371 Date: Jan. 23, 2006

(30) Foreign Application Priority Data
Sep. 27, 2002 (EP) 02021605.7

(51) Int. Cl. C12P 7/26; C12N 1/21; C12N 15/74; C12N 1/18

(52) U.S. Cl. 435/148; 435/471; 435/483; 435/252.3; 435/254.2

(57) ABSTRACT

Disclosed is a process for producing actinol from ketoisophorone which comprises contacting ketoisophorone with a recombinant microorganism or cell-free extract thereof in a reaction mixture, wherein said recombinant microorganism is obtainable by transforming a host microorganism, e.g. selected from the group consisting of microorganisms of the genera Saccharomyces, Zygosaccharomyces, and Candida, such as commercially available baker's yeast, Saccharomyces cerevisiae ATCC7754, Saccharomyces rouxii (Zygosaccharomyces rouxii) HUT7191 (IFO 0494), Saccharomyces delbrueckii HUIT7116 (Saccharomyces unisporus IFO 0298), Saccharomyces delbrueckii (Torulaspora delbrueckii) HUT7102, Saccharomyces willianus HU7106, Zygosaccharomyces bailii ATCC11486, Candida tropicalis IFO 1403, and a mutant thereof, which is capable of reducing ketoisophorone to levodione with a levodione reductase gene, e.g. a levodione reductase gene derived from a microorganism belonging to the genus Corynebacterium, such as C. aquaticum AKU611 (FERM BP-6448) or a mutant thereof, and isolating the produced actinol from the reaction mixture.

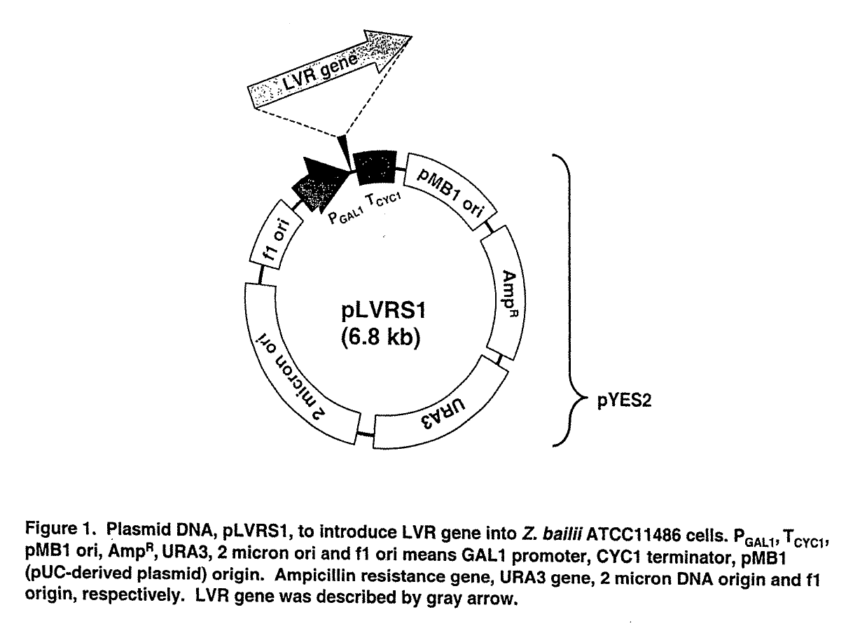

The present invention relates to a one-step process for the microbial production of (4R, 6R)-4-hydroxy-2,2,6-trimethylcyclohexanone (hereinafter referred to as actinol) from 2,6,6-trimethyl-2-cyclohexene-1,4-dione (hereinafter referred to as ketoisophorone) and to recombinant organisms involved in such process.

Actinol is useful for the synthesis of carotenoids, such as zeaxanthin. EP 982,406 discloses an efficient microbial production of actinol which comprises contacting levodione with a microorganism which is selected from the group consisting of microorganisms of the genera Cellulomonas, Corynebacterium, Planococcus, and Arthrobacter, and which is capable of selective asymmetric reduction of levodione to actinol, and recovering the resulting actinol from the reaction mixture. EP 1,026,235 discloses an enzyme, levodione reductase, that acts on levodione to produce actinol, which was isolated from Corynebacterium aquaticum AKU611 (FERM BP-6448). The genetic material such as an isolated DNA comprising a nucleotide sequence coding for an enzyme having levodione reductase activity, a polypeptide encoded by such a DNA, recombinant organisms and the like, are disclosed by EP 1,122,315.

EP 1,074,630 discloses an efficient microbial production of levodione which comprises contacting ketoisophorone with at least one kind of yeast capable of converting ketoisophorone into levodione and selected from the group of species consisting of Saccharomyces rouxii (Zygosaccharomyces rouxii), Saccharomyces delbrueckii (Saccharomyces unisporus, Torulaspora delbrueckii), Saccharomyces willianus, Zygosaccharomyces bailii, and Candida tropicalis. Isolation and characterization of an enzyme ketoisophorone reductase that acts on ketoisophorone to produce levodione was recently reported.

Until now there was no biological one-step process for the direct production of optically active actinol from ketoisophorone, which requires two sequential reactions from ketoisophorone to levodione, and levodione to actinol.

The present invention is directed to a process for the production of actinol from ketoisophorone with high optical purity by a single step reaction using a recombinant micro-organism which is capable of reducing ketoisophorone to levodione, and levodione to actinol simultaneously.

More particularly, the present invention provides a process for producing actinol from ketoisophorone by contacting ketoisophorone with a recombinant microorganism or cell-free extract thereof which has both ketoisophorone-reducing activity and levodione-reducing activity because of introduction of ketoisophorone reductase gene or levodione reductase gene, or both, and isolating the resulted actinol from the reaction mixture.

In another aspect the present invention provides a process for producing actinol from ketoisophorone by contacting ketoisophorone with both ketoisophorone reductase, which is capable of catalyzing the conversion of ketoisophorone to levodione, and levodione reductase, which is capable of catalyzing the conversion of levodione to actinol, simultaneously.

The present invention further provides a recombinant microorganism, that is obtained by transforming a host microorganism which is capable of reducing ketoisophorone to levodione with levodione reductase gene.

More particularly, the present invention provides a recombinant microorganism, that is obtained by transforming a host microorganism which is capable of reducing levodione to actinol with ketoisophorone reductase gene.

In still another aspect the present invention provides a recombinant microorganism which expresses both ketoisophorone reductase gene and levodione reductase gene.

In the present invention, any recombinant microorganism which is capable of producing actinol from ketoisophorone can be used. In the present invention, any microorganism which is capable of producing levodione from ketoisophorone can be used as a host microorganism to obtain a recombinant microorganism which is capable of producing actinol from ketoisophorone by introducing levodione reductase gene in it. Said microorganism can also be used as a donor strain for ketoisophorone reductase gene. A preferred microorganism is selected from the group consisting of microorganisms of the genera Saccharomyces, Zygosaccharomyces, and Candida. Examples include S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, mutants thereof, and even commercially available baker's yeast (available from e.g. the Oriental Yeast Co., Ltd.). These yeasts are disclosed in EP 1,074,630, and have been deposited with at least one of the depositaries American Type Culture Collection (ATCC), 10801 University Boulevard Manassae, Va. 20100-2209, USA; HUT Culture Collection Room, Department of Fermentation Technology, Hiroshima University; and Institute for Fermentation Osaka (IFO), 17-85 Jusohonmachi 2-chome, Yodogawa-ku, Osaka, Japan. Each is available from the pertinent depositary to anyone upon request.

In the present invention, any microorganism which is capable of producing actinol from levodione can be used as a host microorganism to obtain a recombinant microorganism which is capable of producing actinol from ketoisophorone by introducing ketoisophorone reductase gene in it. Said microorganism can also be used as a donor strain for levodione reductase gene. A preferred microorganism is selected from the group consisting of microorganisms of the genera Cellulomonas, Corynebacterium, Planococcus, and Arthrobacter, including Cellulomonas sp. AKU672 (FERM BP-6449), Corynebacterium aquaticum AKU610 (FERM BP-6447), C. aquaticum AKU611 (FERM BP-6448), P. okeanokoites AKU152 (IFO 15880), A. sulfureus AKU635 (IFO 12678), and mutants thereof. These microorganisms are disclosed in EP 982,406. Cellulomonas sp. AKU672 (FERM BP-6449), Corynebacterium aquaticum AKU610 (FERM BP-6447) and C. aquaticum AKU611 (FERM BP-6448) were deposited at the National Institute of Advanced Industrial Science and Technology (AIST), Tsukuba Central 6, 1-1-1 Higashi, Tsukuba, Ibaraki 305-8566, Japan, in the name of F. Hoffmann-La Roche AG of Grenzacherstrasse 124, CH-4070 Basle, Switzerland on Aug. 4, 1998, under Budapest Treaty. P. okeanokoites AKU152 (IFO 15880) and A. sulfureus AKU635 (IFO 12678) were deposited at IFO under the deposit No. described above, and any one can publicly obtain them from the depository. One of the most preferred strains is C. aquaticum AKU611.

In the present invention, any microorganism which is originally not capable of producing levodione from ketoisophorone, nor actinol from levodione, can be used as a host micro-organism to obtain a recombinant microorganism which is capable of producing actinol from ketoisophorone by introducing both genes encoding ketoisophorone reductase and levodione reductase in it.

Either a growing or a resting cell culture or immobilized cells or a cell-free extract, or the like, of said recombinant microrganism may be used for the production of actinol. A growing cell culture can be obtained by culturing a recombinant microorganism in a nutrient medium containing saccharides such as glucose or sucrose, alcohols, such as ethanol or glycerol, fatty acids, such as oleic acid and stearic acid or esters thereof, or oils, such as rapeseed oil or soybean oil, as carbon sources; ammonium sulfate, sodium nitrate, peptone, amino acids, corn steep liquor, bran, yeast extract and the like, as nitrogen sources; magnesium sulfate, sodium chloride, calcium carbonate, potassium monohydrogen phosphate, potassium dihydrogen phosphate, and the like, as inorganic salt sources; and malt extract, meat extract, and the like, as other nutrient sources.

Cultivation of the recombinant microorganism can be carried out aerobically or anaerobically at pH values from 4.0 to 9.0, at a temperature in the range of from 10 to 50° C. for 15 min to 72 hours, preferably, at pH values from 5.0 to 8.0, at a temperature in the range of from 20 to 40° C. for 30 min to 48 hours.

Using the growing cell culture thus obtained, a resting cell culture or immobilized cell or cell-free extract may be prepared by any means generally known in the art.

The concentration of ketoisophorone in a reaction mixture can vary depending on other reaction conditions, but, in general, may be between 0.1 g/l and 300 g/l, preferably between 1 g/l and 30 g/l.

In the present invention, actinol can also be produced by contacting ketoisophorone with ketoisophorone reductase and levodione reductase concomitantly.

One of the most preferred levodione reductases and a method for its preparation is described in EP 1,026,235. This enzyme was characterized by the following physicochemical properties:

1) The levodione reductase catalyzes regio- and stereoselective reduction of levodione to actinol.

2) The relative molecular mass of the enzyme is estimated to be 142,000 -155,000±10,000 Da, consisting of four homologous subunits having a molecular mass of 36,000±5,000 Da.

3) The optimum temperature is 15-20° C. at pH 7.0 and the optimum pH is 7.5.

4) The enzyme requires NAD⁺ or NADH as a cofactor and is highly activated by mono-valent cations, such as K⁺, Na⁺, Cs⁺, Rb⁺, and NH₄⁺.

Levodione reductase catalyzes the reduction of levodione to actinol in the presence of a co-factor according to the following formula:
levodione+NADH [symbol] actinol+NAD

Ketoisophorone reductase and its genetic material such as an isolated DNA comprising a nucleotide sequence coding for an enzyme having ketoisophorone reductase activity can be obtained from a microorganism which is capable of reducing ketoisophorone to levodione. A preferred microorganism is selected from the group consisting of microorganisms of the genera Saccharomyces, Zygosaccharomyces and Candida, including S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, mutants thereof, and even commercially available baker's yeast (available from e.g. the Oriental Yeast Co., Ltd.). These microorganisms are disclosed in EP 1,074,630.

The enzyme reaction may, e.g., be performed as follows: The basal reaction mixture (total volume: 1 ml): 200 μl of 1 M potassium phosphate buffer (pH 7.0), 40 μl of 8 mM NADH in 0.2 mM KOH, 200 μl of ketoisophorone solution, and 20-80 μl of the enzyme solution, and water up to 1 ml, is incubated at pH values of from 4.0 to 9.0, at a temperature range of from 10 to 50° C. for 5 min to 48 hours, preferably at pH values of from 5.0 to 8.0, at a temperature range of from 20 to 40° C. for 15 min to 24 hours.

The concentration of ketoisophorone in a reaction mixture can vary depending on other reaction conditions, but, in general, may be between 0.1 g/l and 300 g/l, preferably between 1 g/l and 30 g/l.

Actinol produced biologically or enzymatically in the reaction mixture as described above may be extracted by an organic solvent such as ethyl acetate, n-hexane, toluene, or n-butyl to recover the actinol into the organic solvent layer. The extract is analyzed by known methods such as gas chromatography, high performance liquid chromatography, thin layer chromatography or paper chromatography, or the like.

After the reaction, actinol in the reaction mixture may be recovered, for example, by extraction with a water-immiscible organic solvent which readily solubilizes actinol, such as ethyl acetate, n-hexane, toluene or n-butyl acetate. Further purification of actinol can be effected by concentrating the extract to directly crystallize actinol or by the combination of various kinds of chromatography, for example, thin layer chromatography, adsorption chromatography, ion-exchange chromatography, gel filtration chromatography or high performance liquid chromatography.

The following Examples further illustrate the present invention, but these are not thereby limiting the scope of the invention.

### EXAMPLE 1

Production of Levodione By Reducing Ketoisophorone Using S. cerevisiae INVScI Resting Cells

S. cerevisiae INVScI was inoculated into YP-raffinose medium (5 ml in a test tube) containing 10 g/l of Bacto Yeast Extract, 20 g/l of Bacto Peptone, and 20 g/l of raffinose, and cultivated at 30° C. for 20 hr. A portion of the seed culture was inoculated into the same medium (10 ml in a test tube) and cultivated at 30° C. for 5 hr. Initial optical density value for 600 nm wavelength of the cultivation was adjusted to 0.2. After addition of 20% galactose solution (1 ml), cultivation was continued for 16 hr. The main culture broth of each test tube was collected and harvested by centrifugation. The pellet of the centrifugation was used as a resting cell of the following ketoisophorone-reducing reaction after wash with 0.1 M of KPB (pH 6.0).

The ketoisophorone-reducing reaction was done as follows. 0.11 g of resting cells were suspended into 1 ml of reaction buffer, which is 0.1 M of KPB (pH 6.0) containing 50 g/l of glucose and 5.0 g/l of ketoisophorone, and incubated at 30° C. in a test tube with gently shaking (200 rpm). The reaction solution was recovered by centrifuging the reaction mixture and removing resting cell pellet. Resulting reaction solution was then extracted with equal volume of ethyl acetate and analyzed by gas chromatography [column: BGB-176 (BGB Analytik AG, Switzerland), instrument: GC-14A (SHIMADZU, Japan), carrier gas: He, column temperature: 1° C./min elevation from 100° C. to 150° C., injector temperature: 200° C.]

The result of the reaction is described in Table 1. After 17.0 hr reaction, 2.8 g/l of levodione was produced from 5.0 g/l of ketoisophorone. Concentration of a major by-product, (S,R)-isomer of actinol, was analyzed to be 0.7 g/l.

TABLE 1
Ketoisophorone-reducing reaction using S. cerevisiae INVScI cells

| time | titer (g/l) | | | |
|---|---|---|---|---|
| (hr) | ketoisophorone | (R) -levodione | (R, R) -actinol | (S, R) -actinol |
| 0 | 3.60 | 0.00 | 0.00 | 0.00 |
| 1 | 3.69 | 0.00 | 0.00 | 0.02 |
| 2 | 2.65 | 0.53 | 0.00 | 0.05 |
| 4 | 2.25 | 1.35 | 0.00 | 0.12 |
| 6 | 1.06 | 1.94 | 0.01 | 0.18 |
| 17 | 0.00 | 2.78 | 0.07 | 0.65 |
| 19 | 0.00 | 2.58 | 0.07 | 0.60 |

### EXAMPLE 2

Introduction of the Levodione Reductase Gene Derived From C. aquaticum AKU611 Into S. cerevisiae INVScI

A DNA fragment encoding levodione reductase gene has already been cloned by using PCR primers designed from the information based on partial amino acid sequences of purified levodione reductase as described below.

At first, genomic DNA of Corynebacterium aquaticum AKU611 (FERM BP-6448) was prepared using Genome Isolation Kit (BIO101). Using the prepared genomic DNA as template, a complete coding sequence for the levodione reductase gene without excessive flanking region was obtained by PCR amplification using a thermal cycler (Perkin elmer 2400, U.S.A.). The two synthetic primers used were: LV-ORF(+)(SEQ ID NO:1) (having an EcoRI site GAATTC) and LV-ORF(−) (SEQ ID NO:2) (having an PstI site CTGCAG). The PCR mixture (0.02 ml) contained 5 pmol of each primer, 0.2 mM of each dNTP, and 1 U of LA Taq (Takara Shuzo co.LTD/Kyoto, Japan). The initial template denaturation step consisted of 1 min at 94° C. An amplification cycle of 20 sec at 98° C., 2 min at 70° C. and 4 min at 72° C. was repeated for 25 times.

By this reaction, a DNA fragment containing a complete ORF of the levodione reductase gene (0.8 Kb) was amplified. This amplified levodione reductase gene was treated with EcoRI and PstI, and ligated with a vector, pKIC223-3 (Amersham Bioscience I Buckinghamshire, England) that was predigested with EcoRI and PstI to construct a plasmid, pKKLR(1-15). E. coli JM109 was transformed with the ligation mixture, and several clones were selected for sequence analysis. The sequence of the cloned levodione reductase gene of each candidate clone was examined. One of the clones that showed completely the same sequence as the levodione reductase sequence of Corynebacterium aquaticum AKU611 (FERM BP-6448) was named as JM109[pKKLR(1-15)]. pKKLR(l-15) was isolated from this strain and used for further experiments.

The 0.8 kb DNA fragment encoding levodione reductase gene was cut out from plasmid pKKLR(1-15) which is comprising levodione reductase gene and E. coli vector pKK223-3, and inserted into pYES2, which is an expression vector for S. cerevisiae. GAL1 promoter and CYC1 terminator, both of which are originally existing on pYES2, are connected to the levodione reductase gene on the plasmid as a result of ligation of the 0.8 kb levodione reductase fragment with EcoRI-digested form of the plasmid pYES2, followed by blunting of the ligated DNA and self-ligation of the DNA. After confirming the direction of the inserted levodione reductase gene, this plasmid, named as pLVRS1, was used to transform S. cerevisiae INVScI. The plasmid is illustrated in the Figure, depicting plasmid DNA, pLVRS1, to introduce LVR gene into Z. bailii ATCC11486 cells. P_{GAL1}, T_{CYC1}, pMB1 ori, AmP^{R}, URA3, 2 micron ori and fl ori means GAL1 promoter, CYC1 terminator, pMB1 (pUC-derived plasmid) origin, ampicillin resistance gene, URA3 gene, 2 micron DNA origin and fl origin, respectively. LVR gene is described by gray arrow. The levodione reductase-encoding plasmid pLVRS1 was introduced into S. cerevisiae INVScI cells by using S. c. EasyComp Transformation Kit (Invitrogen). The method of the transformation was basically the same as the protocol of the transformation kit. Transformed cells can be selected by spreading resulting transformation solution onto solid synthetic medium which lacks uracil because of the presence of the uracil auxotroph of the parent strain and URA3 marker of the plasmid, and incubating these plates for 4 days at 30° C. Presence of the plasmid in the yeast strain was confirmed by checking occurrence of pLVRS1-containing E. coli DH5 alpha strains after transformation of the E. coli strain by using total DNA of the candidate of S. cerevisiae INVScI transformant strain as a donor DNA. The strain possessing PLVRS1, S. cerevisiae (pLVRS1), was then used for further experiments.

### EXAMPLE 3

Production of Actinol By Reducing Ketoisophorone Using S. cerevisiae INVScI Resting Cells Containing the Levodione Reductase Gene Derived From C. aquaticum AKU611

The ketoisophorone-reducing reaction using resting cells of S. cerevisiae INVScI possessing pLVRS1 was originally performed by basically the same method as described in Example 1. The result of the reaction is described in Table 2. After 19.0 hr reaction, 1.6 g/l of actinol was produced from 5.0 g/l of ketoisophorone. Optical purity for actinol was not so high (67.2%) because of the relatively high value of (S,R)-isomer concentration (0.48 g/l).

TABLE 2
Ketoisophorone-reducing reaction using
S. cerevisiae INVScI possessing pLVRS1

| Time | titer (g/l) | | | | | optical purity for |
|---|---|---|---|---|---|---|
| (hr) | KIP | (R)-LDN | (R, R)-ACT | (S, R)-ACT | (S)-PHO | (R, R)-ACT (% e.e) |
| 0 | 3.79 | 0.00 | 0.00 | 0.00 | 0.00 | |
| 1 | 3.02 | 0.00 | 0.03 | 0.01 | 0.33 | 66.6 |
| 2 | 2.44 | 0.39 | 0.12 | 0.03 | 0.52 | 73.0 |
| 4 | 1.61 | 0.83 | 0.31 | 0.07 | 0.62 | 77.8 |
| 6 | 1.00 | 1.12 | 0.55 | 0.12 | 0.60 | 78.8 |
| 17 | 0.11 | 1.24 | 1.31 | 0.36 | 0.29 | 71.0 |
| 19 | 0.06 | 1.72 | 1.60 | 0.48 | 0.27 | 67.2 |

KIP: ketoisophorone;
(R)-LDN: (R) -levodione;
(R, R)-ACT: (R, R)-actinol;
(S, R)-ACT: (S, R)-actinol,
(S)-PHO: (S)-phorenol

0.33 g of resting cells were suspended into 1 ml of reaction buffer, which was 0.1 M of KPB (pH 6.0) containing 150 g/l of glucose and 10.0 g/l of ketoisophorone, and incubated at 20° C. in a test tube with gently shaking (200 rpm). The reaction solution was recovered, extracted, and analyzed by gas chromatography as described in Example 1.

The result of the reaction is described in Table 3. After 25.0 hr reaction, 3.3 g/l of actinol was produced from 10.0 g/l of ketoisophorone. Since the concentration of (S,R)-isomer could be suppressed to 0.52 g/l, optical purity for actinol was increased to 81.5%.

TABLE 3
Ketoisophorone-reducing reaction using
S. cerevisiae INVScI possessing pLVRS1

| Time | titer (g/l) | | | | | optical purity for |
|---|---|---|---|---|---|---|
| (hr) | KIP | (R)-LDN | (R, R)-ACT | (S, R)-ACT | (S)-PHO | (R, R)-ACT (% e.e) |
| 0 | 11.23 | 0.00 | 0.00 | 0.00 | 0.00 | |
| 1.5 | 6.78 | 0.00 | 0.09 | 0.00 | 0.99 | 100.0 |
| 3.0 | 5.93 | 0.57 | 0.35 | 0.06 | 1.88 | 81.6 |
| 4.5 | 3.86 | 0.86 | 0.65 | 0.09 | 2.10 | 85.9 |
| 6.0 | 2.80 | 0.99 | 0.94 | 0.12 | 2.18 | 85.2 |
| 7.5 | 2.27 | 1.20 | 1.14 | 0.14 | 2.08 | 85.3 |
| 9.0 | 1.77 | 1.23 | 1.34 | 0.16 | 2.06 | 85.4 |
| 22.0 | 0.35 | 1.63 | 3.20 | 0.47 | 1.27 | 82.6 |
| 23.5 | 0.31 | 1.64 | 3.29 | 0.50 | 1.18 | 81.9 |
| 25.0 | 0.25 | 1.51 | 3.34 | 0.52 | 1.08 | 81.5 |
| 26.5 | 0.21 | 1.42 | 3.33 | 0.56 | 0.99 | 80.3 |

KIP: ketoisophorone;
(R)-LDN: (R) -levodione;
(R, R)-ACT: (R, R)-actinol;
(S, R)-ACT: (S, R)-actinol,
(S)-PHO: (S)-phorenol

1. A process for producing actinol from ketoisophorone which comprises contacting ketoisophorone with a recombinant microorganism or cell-free extract thereof in a reaction mixture, wherein said recombinant microorganism is obtainable by transforming a host microorganism, e.g. selected from the group consisting of microorganisms of the genera Saccharomyces, Zygosaccharomyces, and Candida, such as commercially available baker's yeast, Saccharomyces cerevisiae ATCC7754, Saccharomyces rouxii (Zygosaccharomyces rouxii) HUT7191 (IPO 0494), Saccharomyces delbrueckii HUT7116 (Saccharomyces unisporus IFO 0298), Saccharomyces delbrueckii (Torulaspora delbrueckii) HUT7102, Saccharomyces willianus HUT7106, Zygosaccharomyces bailii ATCC11486, Candida tropicalis IFO 1403, and a mutant thereof, which is capable of reducing ketoisophorone to levodione with a levodione reductase gene, e.g. a levodione reductase gene derived from a microorganism belonging to the genus Corynebacterium, such as C. aquaticum AKU611 (FERM BP-6448) or a mutant thereof, and isolating the produced actinol from the reaction mixture.

2. The process according to claim 1, wherein the reaction is carried out at pH values of from 4.0 to 9.0, preferably from 5.0 to 8.0, and at a temperature range from 10 to 50° C., preferably from 20 to 40° C., and for 15 minutes to 72 hours, preferably for 30 minutes to 48 hours.

3. A process for producing actinol from ketoisophorone which comprises contacting ketoisophorone with a recombinant microorganism or cell-free extract thereof in a reaction mixture, wherein said recombinant microorganism is obtainable by transforming a host microorganism, e.g. selected from the group consisting of microorganisms of the genera Cellulomonas, Corynebacterium, Planococcus, and Arthrobacter, such as Cellulomonas sp. AKU672 (FERM BP-6449), Corynebacterium aquaticum AKU610 (FERM BP-6447), C. aquaticum AKU611 (FERM BP-6448), P. okeanokoites AKU152 (IFO 15880), A. sulfureus AKU635 (IFO 12678), and a mutant thereof, which is capable of reducing levodione to actinol with a ketoisophorone reductase gene, e.g. derived from a microorganism belonging to the genera Saccharomyces, Zygosaccharomyces, or Candida, such as commercially available baker's yeast, S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, and a mutant thereof, and isolating the resulted actinol from the reaction mixture.

4. The process according to claim 3, wherein the reaction is carried out at pH values of from 4.0 to 9.0, preferably from 5 to 8.0, and at a temperature range from 10 to 50° C., preferably from 20 to 40° C., and for 15 minutes to 72 hours, preferably for 30 minutes to 48 hours.

5. A process for producing actinol from ketoisophorone which comprises contacting ketoisophorone with a recombinant microorganism or cell-free extract thereof in a reaction mixture, wherein said recombinant microorganism, e.g. selected from the group consisting of microorganisms of the genera Cellulomonas, Corynebacterium, Planococcus, and Arthrobacter, such as Cellulomonas sp. AKU672 (FERM BP-6449), Corynebacterium aquaticum AKU610 (FERM BP-6447), C. aquaticum AKU611 (FERM BP-6448), P. okeanokoites AKU152 (IFO 15880), A. sulfureus AKU635 (IFO 12678), and a mutant thereof, expresses both ketoisophorone reductase gene and levodione reductase gene, e.g. a levodione reductase gene derived from a microorganism belonging to the genus Corynebacterium, such as C. aquaticum AKU611 (FERM BP-6448) or a mutant thereof, and isolating the produced actinol from the reaction mixture.

6. The process according to claim 5, wherein the reaction is carried out at pH values of from 4.0 to 9.0, preferably from 5.0 to 8.0, and at a temperature in the range of from 10 to 50° C., preferably from 20 to 40° C., and for 15 minutes to 72 hours, preferably for 30 minutes to 48 hours.

7. A process for producing actinol from ketoisophorone by contacting ketoisophorone with purified ketoisophorone reductase, e.g. derived from a microorganism belonging to the genera Saccharomyces, Zygosaccharomyces, or Candida, such as commercially available baker's yeast, S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, and a mutant thereof, which is capable of catalyzing the conversion of ketoisophorone to levodione and purified levodione reductase, e.g. a levodione reductase derived from a microorganism belonging to the genus Corynebacterium, such as C. aquaticum AKU611 (FERM BP-6448) or a mutant thereof, which is capable of catalyzing the conversion of levodione to actinol simultaneously.

8. The process according to claim 7, wherein the reaction is carried out at pH values of from 4.0 to 9.0, preferably from 5.0 to 8.0, at a temperature in the range of from 10 to 50° C., preferably from 20 to 40° C., and for 5 minutes to 48 hours, preferably for 15 minutes to 24 hours.

9. A recombinant microorganism that is obtainable by transforming a host organism, e.g. a microorganism belonging to the genera Saccharomyces, Zygosaccharomyces, or Candida, such as commercially available baker's yeast, S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, and a mutant thereof, which is capable of reducing ketoisophorone to levodione with a levodione reductase gene, e.g. a levodione reductase gene derived from a microorganism belonging to the genus Corynebacterium, such as C. aquaticum AKU611 (FERM BP-6448) or a mutant thereof.

10. A recombinant microorganism that is obtainable by transforming a host organism, e.g. a microorganism of the genera Cellulomonas, Corynebacterium, Planococcus, and Arthrobacter, such as Cellulomonas sp. AKU672 (FERM BP-6449), Corynebacterium aquaticum AKU610 (FERM BP-6447), C. aquaticum AKU611 (FERM BP-6448), P. okeanokoites AKU152 (IFO 15880), A. sulfureus AKU635 (IFO 12678), and a mutant thereof, which is capable of reducing levodione to actinol with ketoisophorone reductase gene, e.g, derived from a microorganism belonging to the genera Saccharomyces, Zygosaccharomyces, or Candida, such as commercially available baker's yeast, S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, and a mutant thereof.

11. A recombinant microorganism which expresses both ketoisophorone reductase gene, e.g, derived from a microorganism belonging to the genera Saccharomyces, Zygosaccharomyces, or Candida, such as commercially available baker's yeast, S. cerevisiae ATCC7754, S. rouxii (Z. rouxii) HUT7191 (IFO 0494), S. delbrueckii HUT7116 (S. unisporus IFO 0298), S. delbrueckii (Torulaspora delbrueckii) HUT7102, S. willianus HUT7106, Z. bailii ATCC11486, C. tropicalis IFO 1403, and a mutant thereof, and a levodione reductase gene, e.g. a levodione reductase gene derived from a microorganism belonging to the genus Corynebacterium, such as C. aquaticum AKU611 (FERM BP-6448) or a mutant thereof.
